(19) United States
(12) Patent Application Publication
(10) Pub. No.: US 2014/0336553 A1
(43) Pub. Date: Nov. 13, 2014

(54) ACTIVE KNEE ORTHOSIS

(71) Applicants: André De Cortanze, Bandol (FR); Roger Lecurieux-Clerville, Marseille (FR)

(72) Inventors: André De Cortanze, Bandol (FR); Roger Lecurieux-Clerville, Marseille (FR)

(21) Appl. No.: 13/261,903

(22) PCT Filed: Nov. 15, 2012
(86) PCT No.: PCT/FR2012/052636

§ 371 Date: May 23, 2014

(30) Foreign Application Priority Data
Nov. 23, 2011 (FR) 1160709

(51) Int. Cl. A61F 5/01

(52) U.S. Cl. CPC A61F 5/0125; A61F 2005/0139
USPC 602/16

(57) ABSTRACT

A knee orthosis includes two parts, upper and lower, each provided with at least one attachment means linking supporting parts, suitable for attaching said upper and lower parts respectively to a thigh and to a leg. Both parts are linked via bi-centric articulation means suitable for enabling each of said parts to pivot about a common axis between the ends of the supporting parts and an intermediate part forming a connection rod. A cord linking links the common axis between the connection rod connecting an articulation means to the upper portion of the orthosis, with the distal end of an arm forming one of the supporting parts of the lower portion of the orthosis.

### BACKGROUND

The present invention relates to an active knee orthosis comprising two parts which are articulated with respect to each other and which, being each provided with at least one attachment means, are fixed respectively to a thigh and to a leg of a lower limb of a person or patient in order, on the one hand, to externally strengthen the knee, maintain the joint in place and alleviate the osteoarticular pathologies, and, on the other hand, to correct an abnormal lateral deviation of the curvature of the lower limb, for example of the valgus or varus type, hence the term active.

### SUMMARY

The technical field is the production and the use of such an orthosis or external orthopedic appliance for the knee, which can be fitted in place by the actual wearer.

Many appliances are known in this field, most of them having been the subject of filed patent applications. For example, mention may be made of FR 2 872 205 which was filed by the company Axmed and which describes an orthosis, of which the parts articulated with respect to each other each have lateral rigid side supports connected to each other by a rigid frame, and of which at least one of the side supports is adjustable in length such that, with the side supports thus being able to have different lengths, the corresponding part of the orthosis has a lateral deviation able to correct a valgus-varus.

The patent application UK 2 136 294 filed by the company Northern Scient Equipment also describes an orthosis for a person who suffers from knock knees, comprising a telescopic device arranged on one side of the knee and combined with straps which surround the knee from the other side and thus force the latter toward the telescopic device in order to correct the abnormal deviation of the knee.

Although all of these appliances, and indeed others, allow knee problems to be corrected or alleviated, they are either uncomfortable, especially in the flexion position, or their action is only partial and/or they may even cause side effects that damage the joint.

The problem addressed is therefore to produce an orthopedic appliance or knee orthosis which corrects abnormal lateral deviations of the knee, while at the same time strengthening the joint, and which alleviates the interosseous crushing pressure between the bones of the knee in the extension position, which is comfortable for the patient, especially in the flexion position, and whose corrective and strengthening action on the knee is as complete as possible without causing side effects.

A solution to the problem addressed is a knee orthosis comprising two parts, the first being the upper part and the second being the lower part, said parts each being provided at least with an attachment means which links support components and is suitable for fixing said parts respectively to a thigh and to a leg of a lower limb of a person presenting an abnormal initial lateral deviation, and having a circular general shape that is adaptable respectively to said thigh and to said leg and with the same axis respectively as those, and such that they thus follow the movement of this thigh and of this leg; said two parts are connected via bi-centric articulation means which are intended to be positioned laterally on each side of the knee and are designed to allow the upper part and lower part to each pivot about a common axis between the ends of the support components thereof and an intermediate component forming a connection rod of the corresponding articulation means; according to the invention, said orthosis has a cable linking the common axis, between the connection rod of the articulation means, corresponding to the side of the orthosis opposite the initial deviation, and the upper part of the orthosis, to the distal end of an arm forming one of the support components of the lower part of the orthosis and arranged in a direction almost parallel to the axis thereof: the length of the cable is determined such that it exerts a given traction on the attachment points of its two ends when the upper part and lower part are pivoted in relation to each other in the sense of tending toward the alignment of their axes and, by contrast, is untensioned when the upper part and lower part are pivoted in the opposite sense.

In a preferred embodiment, a first articulation means, corresponding to the side of the orthosis toward which the initial deviation inclines, on the one hand spaces apart the two common axes, between its connection rod and the support components of the upper part and lower part, when these are pivoted in the sense of tending toward the alignment of their axes, and, on the other hand, brings these two common axes together when said upper and lower parts are pivoted in the sense of increasing the relative angle between their axes.

In a particular embodiment, the second articulation means, corresponding to the side of the orthosis opposite the initial deviation, has a pusher component which advances toward the first articulation means, situated on the other side of the orthosis, when the upper part and lower part are pivoted in the sense of tending toward the alignment of their axes, and which moves away from this first articulation means when said upper part and lower part are pivoted in the sense of increasing the relative angle between their axes.

The result is a novel active knee orthosis which solves the problem addressed, on the one hand by correcting the abnormal lateral deviations of the knee and by alleviating the interosseous crushing pressure, which may even involve contact between the bones of the knee, in the extension position of the joint, and, on the other hand, by relaxing the forces used for these corrective and alleviating actions in the flexion position of the joint, which ensures comfortable use for the patient wearing the orthosis.

Moreover, the combination of the three different devices described above, and set out in detail in the description below, permits a corrective, strengthening and pressure-alleviating action which is more complete and more effective than in all the orthoses known to date: and as has been indicated above, these three devices in combination play their role in the extension position of the knee only between 0 and approximately 35°, and they relax their pulling and pushing forces beyond this, allowing the knee to recover a position that is comfortable for the patient in its flexion position.

The advantages mentioned above thus prove the interest of this novel orthosis or knee brace, of which the description and the attached figures provide an illustrative embodiment.

Other embodiments are possible, however, within the scope of the present invention.

### BRIEF DESCRIPTION OF THE DRAWINGS

FIG. 1 is a front cross-sectional view of a knee in the extension position, in which, as a consequence of osteoarticular deficiency, one side of the femoral condyle is in contact with the tibial plateau, creating a lateral imbalance and an abnormal deviation of the joint and causing pain.

FIGS. 2 are cross-sectional views of the knee from FIG. 1 equipped with an orthosis according to the invention, on the one hand in the extension position, corrected by virtue of the prosthesis, and, on the other hand, in the flexion position.

FIGS. 3 are side views and a perspective view of an illustrative embodiment of a first bi-centric lateral articulation means of the orthosis according to the invention, able to move the upper and lower parts thereof away from each other, in the extension position of the knee.

FIGS. 4 are perspective views of an illustrative embodiment of a second bi-centric lateral articulation means of the orthosis according to the invention, able to push against the knee in the extension position thereof.

FIGS. 5 are side and profile views of an illustrative embodiment and of the use of a lateral traction cable of the orthosis according to the invention, in the extension position and flexion position of the knee.

FIGS. 6 are front views of an orthosis according to the invention, in an enlarged representation and in the same positions as in FIGS. 2.

FIGS. 7 are side and profile views of another illustrative embodiment and of the use of a lateral traction cable of the orthosis according to the invention, in the extension position of the knee.

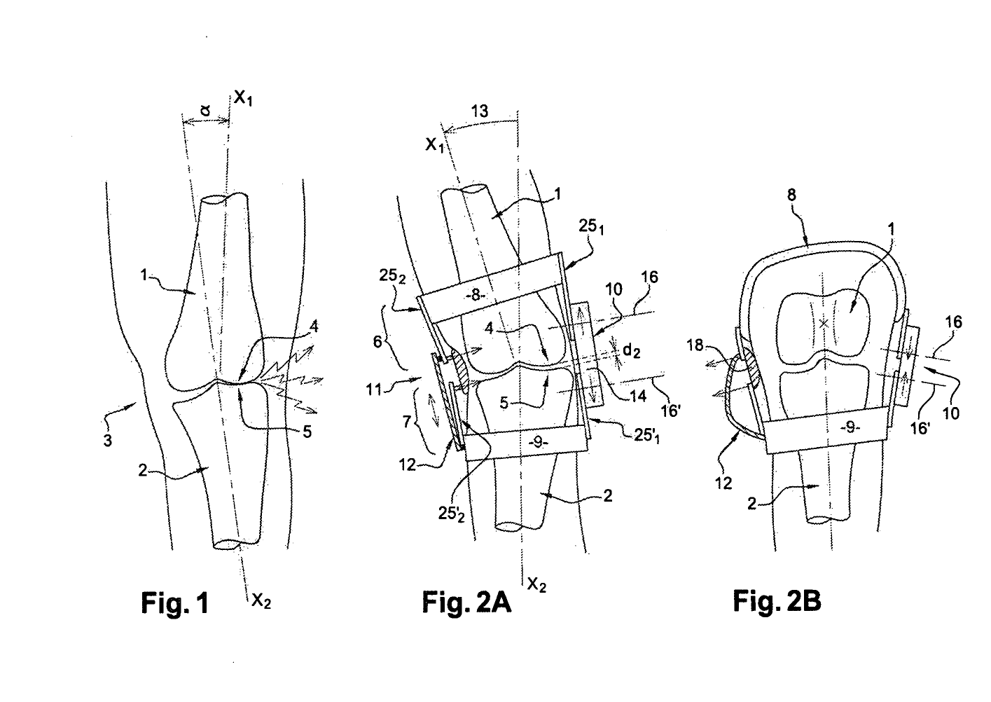

As defined above, the aim of the present invention is to correct the abnormally large initial lateral deviation of angle γ, as shown by way of example in FIG. 1 and representing what is called a bow leg 3, between the axis X₁ of the thigh or of the femur 1 and that X₂ of the leg or of the tibia 2, in the extension position of the knee without orthosis, and to alleviate the pressure, here arising from contact, between the bones of the knee, or between the femoral condyle 4 and the tibial plateau 5.

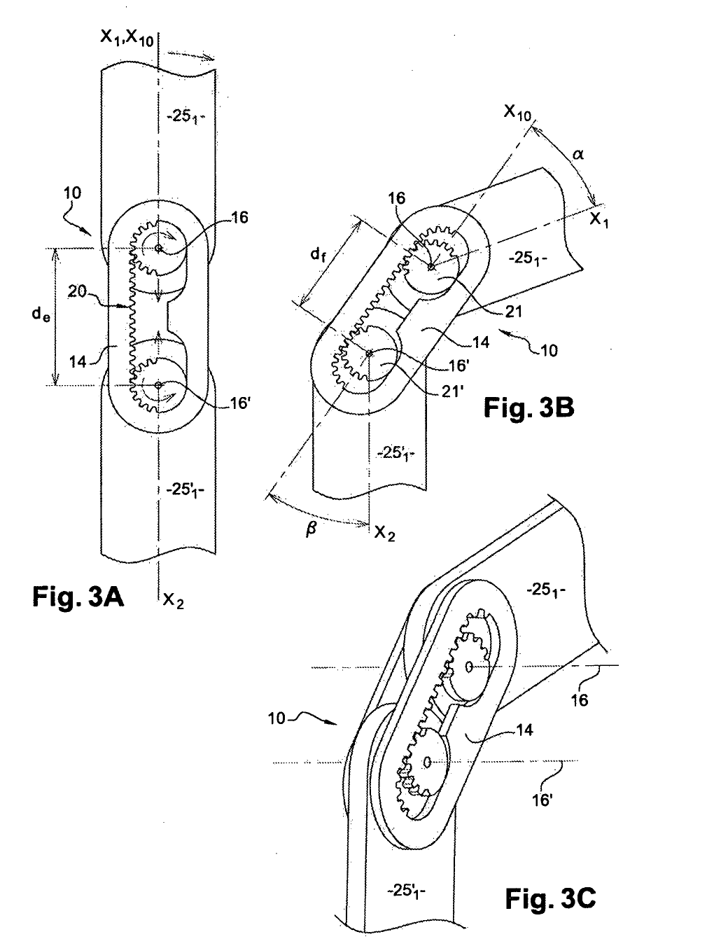

FIG. 2A shows the same knee equipped with the orthosis according to the invention and straightened by the latter from its abnormal lateral deviation y, after rotation 13, albeit shown in an exaggerated manner in this figure for better comprehension. In the flexion position, as shown in FIG. 2B, the relaxation of all the actions of the orthosis on the articulation affords the patient the desired comfort.

### DETAILED DESCRIPTION

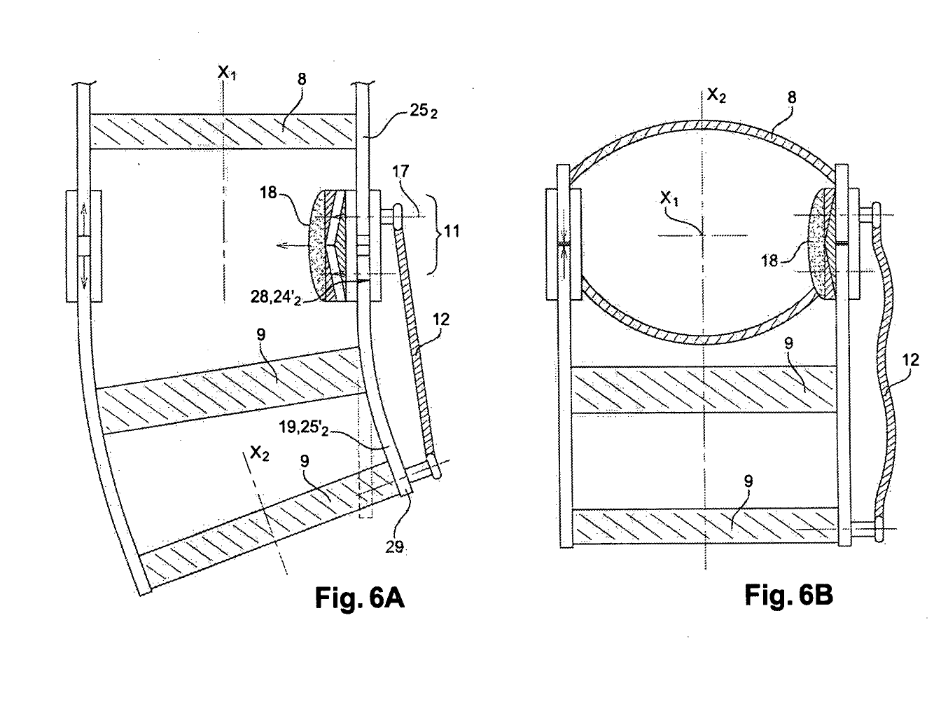

For this purpose, the knee orthosis comprises two parts 6, 7, the first 6 being the upper part and the second 7 being the lower part, said parts each being provided with at least one attachment means such as bases 8, 9 which link support components 25, 25′ and are suitable for fixing said parts 6, 7 respectively to the thigh and to the leg of a lower limb of the person or patient who presents an abnormal lateral deviation γ, as in FIG. 1; these attachment means have a circular general shape that is adaptable respectively to said thigh and to said leg and with the same axis respectively X₁ and X₂ as those; as is depicted schematically in FIG. 6B, this circular general shape is shown by the axial view of the attachment means, which can be a strap 8 on and around the thigh (not shown in this figure) and which is in the flexion position with respect to the leg on which the lower part of the prosthesis is fixed by the two attachment means 9.

Thus, these upper and lower parts 6, 7 of the orthosis according to the invention follow the movements of this thigh and of this leg, and said two parts 6, 7 are connected by bi-centric articulation means 10, 11, which are intended to be positioned laterally on each side of the knee and are designed to allow the upper part 6 and lower part 7 to each pivot about a common axis 16, 16′ for a first articulation means 10 and an axis 17, 17′ for the second articulation means 11, between the ends 24, 24′ of the supports 25, 25′ thereof and an intermediate component forming a connection rod 14, 15 of the corresponding articulation means 10, 11.

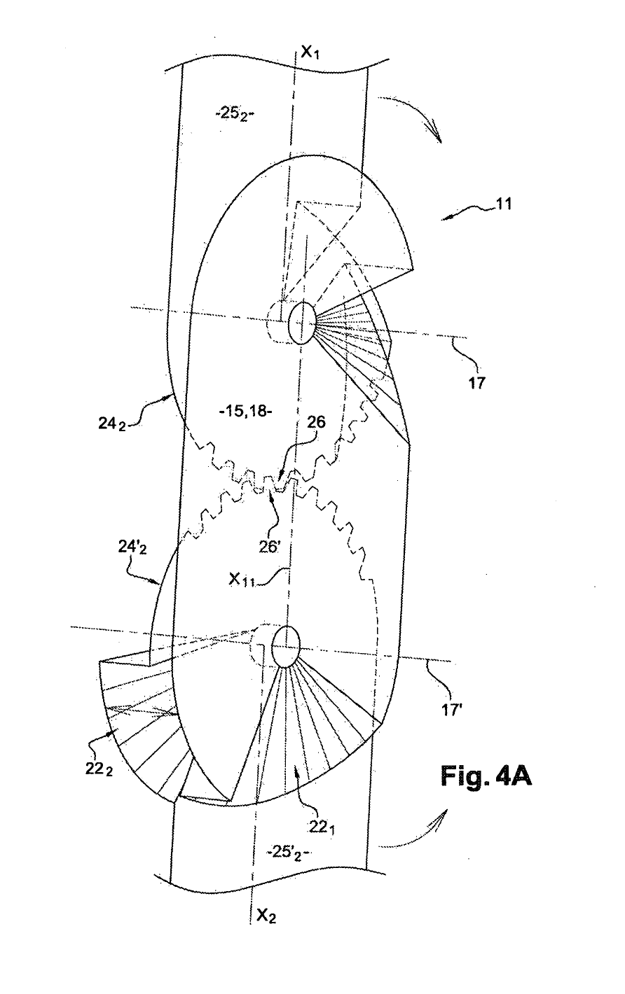

The first articulation means 10, corresponding to the side of the orthosis toward which the initial deviation γ of FIG. 1 inclines, is on the side of the knee which can be described as concave and which is that of the pinching of the articulation, where the tibial plateau 5 is too close to or even in contact with the femoral condyle 4. This first articulation means 10, as shown in FIGS. 2 and 3, on the one hand spaces apart the two common axes 16, 16′, between its connection rod 14 and the support components 25₁, 25′, of the upper part 6 and lower part 7, when these are pivoted in the sense of tending toward the alignment of their axes X, and X₂ and, on the other hand, brings these two common axes together when said upper and lower parts are pivoted in the sense of increasing the relative angle α+β between their axes X, and X₂: the angle α is to be considered between the axis X, of the support component 25₁ and the axis X₁₀ of the connection rod 14, and the angle β between the axis X₂ of the support component 25′₁ and the axis X₁₀ of this connection rod 14.

According to the embodiment in these FIGS. 3, the connection rod 14 has an internal seat which is equipped at least with a rack 20 and in which two toothed wheels 21 cooperating with this rack are guided and turn, said toothed wheels being fixed and each rigidly connected to the end 24, and 24′₁ of a support component 25, 25′₁ respectively of the upper part 6 and lower part 7 of the orthosis.

Thus, as is shown in FIG. 3A in the extension position of the knee, the support components 25, and 25′, are spaced apart from each other to the maximum extent by a maximum spacing “dₑ” of the axes 16 and 16′, whereas, in the position of flexion or semi-flexion as in FIG. 3B, this distance between the axes 16 and 16′ decreases “d_{f}” through the rotation of the toothed wheels 21 along the rack 20, thus bringing the support components 25₁ and 25′₁ closer together and thereby relieving the joint.

Thus, by way of example, the difference in spacing between the axes 16 and 16′ (dₑ−d_{f}) is from 4 to 8 mm in order to obtain an osseous spacing d₂ (according to FIG. 2A), between the edges of the tibial plateau 5 and of the femoral condyle 4, of 2 to 4 mm taking into account the distraction of the “soft” parts such as the skin and muscles.

In another embodiment not shown in an attached figure, the connection rod 14 can have an internal seat equipped with two cavities, in each of which is guided and turns a cam which is fixed and rigidly connected to the end 24₁, 24′₁ of a support component respectively of the upper part 6 and lower part 7 of the orthosis, each cam bearing on the internal shape of its ramp-shaped cavity makes it possible, during its rotation, to obtain the same effects of spacing apart and bringing together as in the embodiment in FIGS. 3.

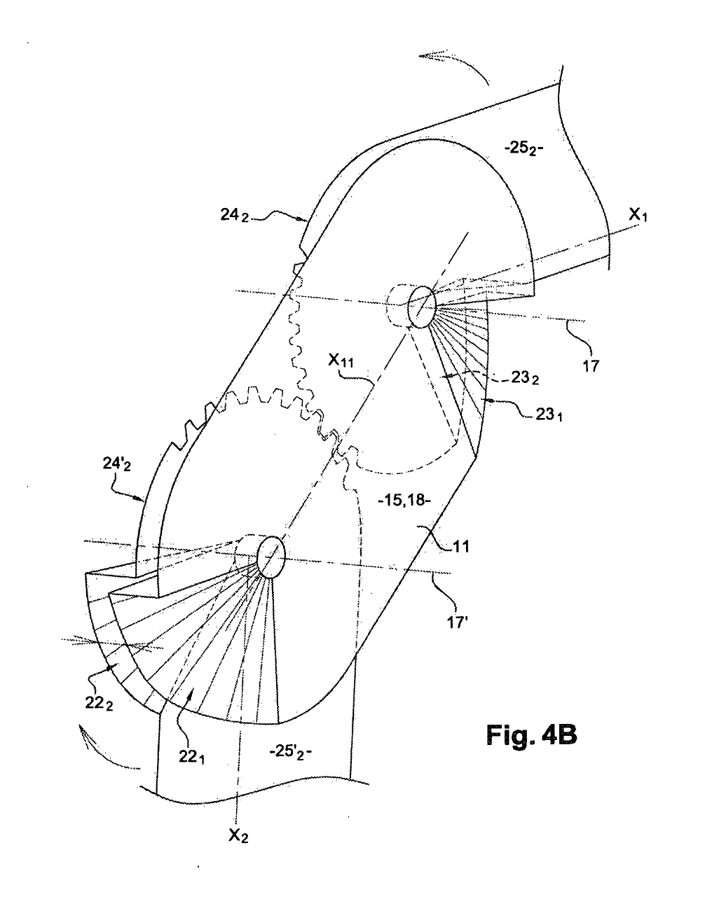

The second articulation means 11, which is positioned on the side of the orthosis opposite the initial deviation γ of FIG. 1, is on the other side of the knee in relation to the side on which the first articulation means 10 is arranged, that is to say on the convex side of the knee corresponding to the spacing apart of the joint where the tibial plateau 5 is too far from the femoral condyle 4. This second articulation means has a pusher component 18 which advances toward the first articulation means 10, situated therefore on the other side of the orthosis, when the upper part 6 and lower part 7 are pivoted in the sense of tending toward the alignment of their axes X₁ and X₂, i.e. in the position of extension of the knee, and which moves away from this first articulation means when said upper part 6 and lower part 7 are pivoted in the sense of increasing the relative angle α+β between their axes X₁ and X₂, i.e. in the flexion position of the knee: the angles α and β are those already defined above, between the axis X₁₁ of the connection rod 15 of this second articulation means 11 and respectively the axes X₁ and X₂ of the upper part 6 and lower part 7, or corresponding support components 25₂ and 25′₂, as shown in FIGS. 4A and 4B.

FIGS. 4A and 4B show an illustrative embodiment of this second articulation means 11 in which the pusher component 18 forms the connection rod 15 of the articulation: the surface of this connection rod 15 is of helicoidal shape over at least a quarter of its surface 22₁, 23₁ around these two axes 17, 17′, which shapes 22, 23 constitute ramps which bear on and cooperate with ends 24₂, 24′₂ of the support components 25₂, 25′₂ fixed respectively to the upper and lower parts of the orthosis and of likewise helicoidal shape 22₂, 23₂ likewise forming a ramp of the same pitch and with the same common axis 17, 17′; said ends 24₂, 24′₂ of the support components each having a portion of a toothed ring 26, 26′ of the same axis 17, 17′, which cooperate with each other.

Thus, in the flexion position of the knee as shown in FIG. 4B, the helicoidal shapes or ramps of the connection rod 15 are entirely imbricated in those of the support components 25₂, 25′₂, and the connection rod 15 is thus in a position closest to the ends 24₂ and 24′₂ of these support components; whereas in the view in FIGS. 4A, in the extension position of the knee joint, the support components 25₂ and 25′₂ having pivoted with respect to the connection rod 15, the helicoidal shapes, bearing on each other by turning as if one were unscrewing them from each other, space apart the connection rod 15 from the ends 24₂, 24′₂ of the support components to the maximum extent: this spacing translates into a pushing effect against the knee, since the support components 25₂, 25′₂ are fixed on the thigh and the leg and cannot move away from each other, and it is therefore the connection rod 15 that plays the role of the pusher component 18, as is shown in FIGS. 2A and 6A.

In addition to the combination of the two articulation means 10, 11 as have been described above, the orthosis according to the invention has a cable 12 linking the common axis 17, between the connection rod 15 of the articulation means 11 and the support component 25₂ of the upper part 6 of the orthosis, to an attachment point 30 fixed on the distal end 29 of an arm 19 forming one of the support components 25′₂ of the lower part 7 of the orthosis and arranged in a direction almost parallel to the axis X₂ thereof.

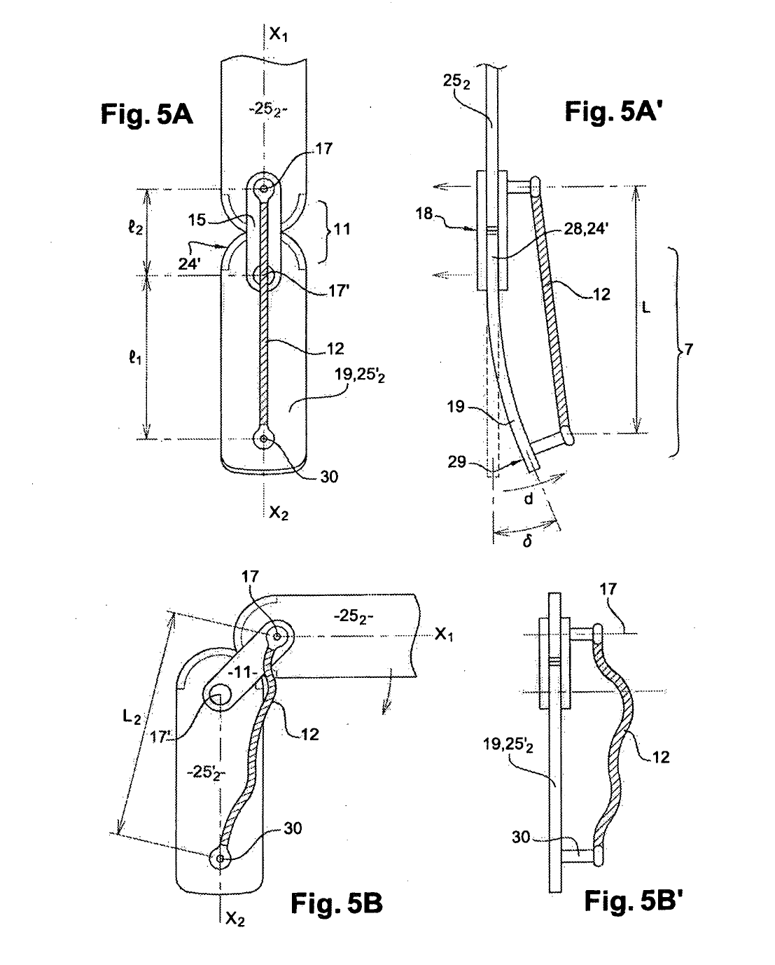

As is shown in FIGS. 5A, 5A′, 6A and 7, the length “L” of the cable 12 is determined such that it exerts a given traction on the attachment points of its two ends when the upper part 6 and lower part 7 are pivoted in relation to each other in the sense of tending toward the alignment of their axes X₁, and X₂, that is to say when the knee joint is in the extension position, and, by contrast, as is shown in FIGS. 5′A, 5′B and 6B, it is untensioned when the upper part 6 and lower part 7 are pivoted in the opposite sense, that is to say when the knee joint is in the flexion position: by way of example, if I₂, which is the distance between the two common axes 17, 17′ of the connection rod 15 and I₁ the distance between the common axis 17′ of the connection rod to the other attachment point 30 of the cable 12, this can have a length L=147.5 mm, for I₂=50 mm and I₁=100 mm; the difference of 2.5 mm between L and I₁+I₂ creates a tensile force in the arm 19 in the extension position: this then moves away from its rest position in order to compensate for this difference, i.e. with the values above, by a distance “d” at its distal end corresponding to an angle δ of about 7°.

According to the embodiments in FIGS. 5 and 6, the arm 19 is made of resilient material with a thinner cross section at its proximal end 28 than at its distal end 29, and its proximal end 28 is integrated in the second articulation means 11 in which it is fixed and pivots around their common axis 17′ with the connection rod 15 when the knee flexes as shown in FIG. 5B; in this position, the length L₂ between the two attachment points 17, 30 of the cable 12 is less than the length L of the latter which is then untensioned, relaxing its stress on the orthosis and thus on the knee.

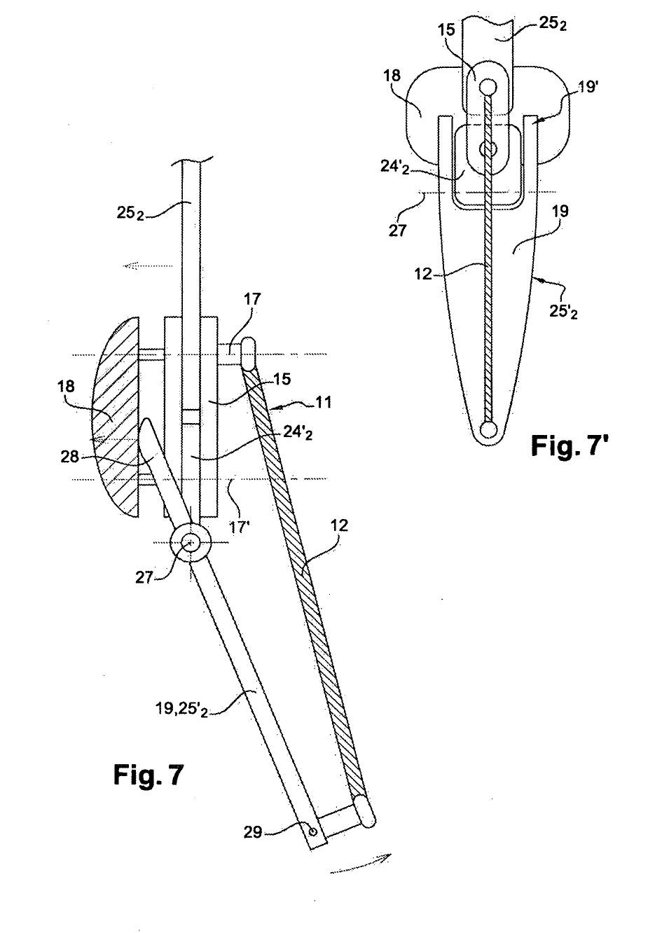

In another embodiment as shown in FIGS. 7 and 7′ in a profile view and a side view, the arm 19 is rigid and articulated about an axis 27 perpendicular to the common axis 17′ of the connection rod 15 with the end 24′₂ of the support component 25′₂: this component is thus composed itself of two parts, the part constituting the arm 19 then having its proximal end 28 distinct from the end 24′₂ and pivoting with the whole of the arm in order to move a pusher component 18 which advances toward the first articulation means 10 situated on the other side of the orthosis, in fact against the knee, when the upper part 6 and lower part 7 are pivoted in the sense of tending toward the alignment of their axes X₁, X₂, as has been described above for other embodiments, and which moves away from this first articulation means when said upper and lower parts are pivoted in the sense of increasing the relative angle α+β between their axes X₁ and X₂: thus, in this embodiment according to FIGS. 7 and 7′, the cable 12 not only performs the function of traction force on the orthosis and thus on the side of the knee, as in the embodiment in FIGS. 5 and 6, but also the function of pushing the second articulation means 11, of which the pusher component 18 forms part.

Whatever the embodiments of the three devices for acting on the knee as described above, the combination of the effects of these three devices, which are only active in the extension position of the knee, i.e. when α+β varies from 0 to 35°, on the one hand ensures the correction of the initial lateral deviation and, on the other hand, alleviates the interosseous crushing pressure on the side at which the knee is pinched and which actually causes the deviation. These three devices relax their spacing-apart, pulling and pushing actions beyond 35°, i.e. in the position of flexion, allowing the knee to recover a more comfortable position for the patient.

The combination of these three effects through the complementary nature of the three devices described above, and shown by way of example in the attached figures, ensures maximum efficacy in the straightening of the knee joint, either of the valgus or the varus type.

1. A knee orthosis comprising:
an upper part and
a second lower part,
said parts each being provided with at least one attachment means which links support components and is suitable for fixing said parts respectively to a thigh and to a leg of a lower limb of a person presenting an abnormal initial lateral deviation, and with a circular general shape that is adaptable respectively to said thigh and to said leg and with the same axis respectively (X₁) and (X₂) as those, and such that they thus follow the movement of this thigh and of this leg,
said two parts being connected via bi-centric articulation means which are intended to be positioned laterally on each side of the knee and are designed to allow the upper part and lower part to each pivot about a common axis between the ends of the support components thereof and an intermediate component forming a connection rod of the corresponding articulation means
wherein the orthosis has a cable linking the common axis, between the connection rod of the articulation means, corresponding to the side of the orthosis opposite the initial deviation (γ), and the upper part of the orthosis, to the distal end of an arm forming one of the support components of the lower part of the orthosis and arranged in a direction almost parallel to the axis (X₂) thereof, the length of the cable being determined such that it exerts a given traction on the attachment points of its two ends when the upper part and lower part are pivoted in relation to each other in the sense of tending toward the alignment of their axes (X₁, X₂) and, on the contrary, is untensioned when the upper part and lower part are pivoted in the opposite sense.

2. The knee orthosis of claim 1, wherein a first articulation means, corresponding to the side of the orthosis toward which the initial deviation (γ) inclines, on the one hand spaces apart the two common axes, between its connection rod and the support components of the upper part and lower part, when these are pivoted in the sense of tending toward the alignment of their axes (X₁) and (X₂), and, on the other hand, brings these two common axes together when said upper and lower parts are pivoted in the sense of increasing the relative angle (α+β) between their axes (X₁) and (X₂).

3. The knee orthosis of claim 2, wherein the connection rod has an internal seat which is equipped at least with a rack and in which two toothed wheels cooperating with this rack are guided and turn, said toothed wheels being fixed and each rigidly connected to the end of a support component respectively of the upper part and lower part of the orthosis.

4. The knee orthosis of claim 2, wherein the connection rod has an internal seat equipped with two cavities, in each of which is guided and turns a cam which is fixed and rigidly connected in each case to the end of a support component respectively of the upper and lower parts of the orthosis, each cam bearing on the internal shape of its ramp-shaped cavity.

5. The knee orthosis claim 1 wherein the second articulation means, corresponding to the side of the orthosis opposite the initial deviation (γ), has a pusher component which advances toward the first articulation means, situated on the other side of the orthosis, when the upper part and lower part are pivoted in the sense of tending toward the alignment of their axes (X₁) and (X₂), and which moves away from this first articulation means when said upper part and lower part are pivoted in the sense of increasing the relative angle (α+β) between their axes (X₁) and (X₂).

6. The knee orthosis of claim 5, characterized that wherein the pusher component forms the connection rod of the articulation, which connection rod is of helicoidal shapes over at least a quarter of its surface around these two axes, which shapes constitute ramps which bear on and cooperate with ends of the support components fixed respectively to the upper and lower parts of the orthosis and of likewise helicoidal shape likewise forming a ramp of the same pitch and with the same common axis, and said ends of the support components each having a portion of a toothed ring with the same axis, which cooperate with each other.

7. The knee orthosis claim 1 wherein the arm is made of resilient material with a thinner cross section at its proximal end than at its distal end, and its proximal end is integrated in the second articulation means, in which it is fixed and pivots around their common axis with the connection rod.

8. The orthosis of claim 1 wherein the arm is rigid and articulated about an axis perpendicular to the common axis of the connection rod with the end of the support component, the latter thus being composed of two parts, the part constituting the arm then having its proximal end distinct from the end and pivoting with the whole of the arm in order to move a pusher component which advances toward the first articulation means situated on the other side of the orthosis when the upper part and lower part are pivoted in the sense of tending toward the alignment of their axes, and which moves away from this first articulation means when said upper and lower parts are pivoted in the sense of increasing the relative angle (α+β) between their axes and.
